(19) United States
(12) Patent Application Publication
(10) Pub. No.: US 2018/0338698 A1
(43) Pub. Date: Nov. 29, 2018

(54) MEASUREMENT APPARATUS AND RECORDING MEDIUM RECORDING MEASUREMENT PROGRAM

(71) Applicant: FUJITSU LIMITED, Kawasaki-shi (JP)

(72) Inventor: Kazuhiro YOSHIMURA, Kawasaki (JP)

(73) Assignee: FUJITSU LIMITED, Kawasaki-shi (JP)

(21) Appl. No.: 16/055,519

(22) Filed: Aug. 6, 2018

Related U.S. Application Data
(63) Continuation of application No. PCT/JP2016/055833, filed on Feb. 26, 2016.

(51) Int. Cl. A61B 5/0452; A61B 5/024; A61B 5/00

(52) U.S. Cl. CPC A61B 5/0452; A61B 5/02444; A61B 5/0006; A61B 5/02405

(57) ABSTRACT

A measurement apparatus includes a biological sensor configured to measure a biological signal of a person to be measured, and a processor configured to: acquire posture information on a posture of the person to be measured, and change accuracy of measurement data of the biological sensor, based on the acquired posture information.

### CROSS-REFERENCE TO RELATED APPLICATION

This application is a continuation application of International Application PCT/JP2016/055833 filed on Feb. 26, 2016 and designated the U.S., the entire contents of which are incorporated herein by reference.

### FIELD

The present disclosure relates to a measurement apparatus and a measurement program.

### BACKGROUND

There is a known technology of calculating biological information (for example, a heartbeat from an electrocardiogram, a pulse rate from a pulse wave).

Related Art is disclosed in Japanese Laid-open Patent Publication No. 2015-112205, Japanese Laid-open Patent Publication No. 2006-312010, or International Publication Pamphlet No. WO 2011/024425.

### SUMMARY

According to one aspect of the embodiments, a measurement apparatus includes a biological sensor configured to measure a biological signal of a person to be measured, and a processor configured to: acquire posture information on a posture of the person to be measured, and change accuracy of measurement data of the biological sensor, based on the acquired posture information.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram schematically illustrating an example of an electrocardiogram monitor system 100.

FIG. 2 is a diagram illustrating a configuration example of an electrocardiogram measurement apparatus 1 according to Embodiment 1.

FIG. 3 is a table chart illustrating an example of learning information.

FIG. 4 is a diagram schematically illustrating a time series of power consumption of an electrocardiogram sensor 12 and the like in a high-accuracy measurement mode.

FIG. 5 is a diagram schematically illustrating the time series of the power consumption of the electrocardiogram sensor 12 and the like in a low-accuracy measurement mode.

FIG. 6 is a sequence diagram illustrating an example of initial setting processing of the electrocardiogram monitor system 100.

FIG. 7 is an explanatory diagram of a time zone success probability table.

FIGS. 8A and 8B are a flowchart illustrating an example of measurement processing performed by the electrocardiogram measurement apparatus 1.

FIG. 9 is an explanatory diagram of a success probability calculating method.

FIG. 10 is an explanatory diagram of another calculation method of success probability.

FIG. 11 is a diagram illustrating an example of a hardware configuration of a wireless control device 21 of an electrocardiogram monitor device 6.

FIG. 12 is a diagram illustrating a configuration example of an electrocardiogram measurement apparatus 1A according to Embodiment 2.

FIG. 13 is a diagram schematically illustrating a time series of power consumption in the electrocardiogram sensor 12 and the like during high-accuracy measurement.

FIG. 14 is a diagram schematically illustrating the time series of the power consumption of the electrocardiogram sensor 12 and the like during a low-accuracy measurement.

FIG. 15 is a diagram illustrating a configuration example of an electrocardiogram measurement apparatus 1B according to Embodiment 3.

### DESCRIPTION OF EMBODIMENTS

For example, a motion of a person to be measured is detected using acceleration, a biological signal is acquired from a biological sensor under fixed measurement conditions, and biological information (for example, a heartbeat from an electrocardiogram, a pulse rate from a pulse wave) is calculated. The motion of the person to be measured is, for example, a stationary state or a motion state, and the fixed measurement conditions are, for example, a sampling frequency and data accuracy, and the biological signal is, for example, measurement data such as an electrocardiogram and a pulse wave. The biological information is, for example, a heartbeat obtained from the electrocardiogram and the pulse rate obtained from the pulse wave.

However, a measurement apparatus may not change accuracy of measurement data of a biological sensor depending on a posture of a person to be measured.

For example, a measurement apparatus and a measurement program that can change accuracy of measurement data of a biological sensor depending on a posture of a person to be measured are provided.

Hereinafter, each embodiment will be described in detail with reference to the accompanying drawings.

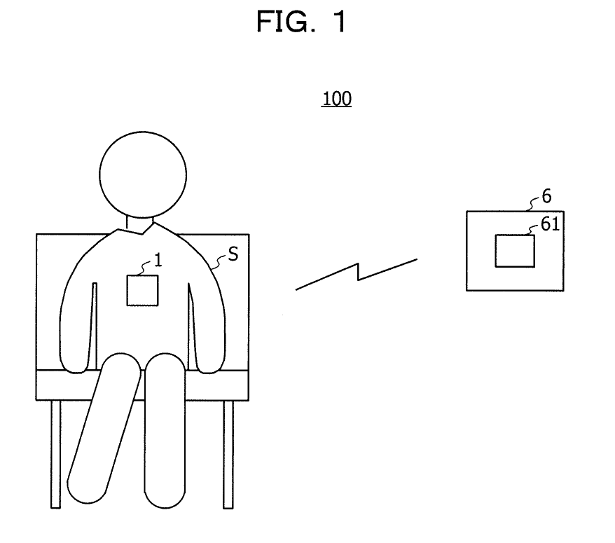

FIG. 1 is a diagram schematically illustrating an example of an electrocardiogram monitor system 100. The electrocardiogram monitor system 100 includes an electrocardiogram measurement apparatus 1 (an example of a measurement apparatus) and an electrocardiogram monitor device 6.

The electrocardiogram measurement apparatus 1 measures an electrocardiogram (an example of a biological signal) of a user S (an example of a person to be measured) and transmits measurement data to the electrocardiogram monitor device 6. The user S is random, but it is preferable for the user to be, for example, a pregnant woman. Hereinafter, it is assumed that the user S is a pregnant woman as an example. Hereinafter, it is assumed that the electrocardiogram of the user S includes an electrocardiogram of a fetus in the body of the user S, unless described in particular. In addition, when distinguishing between these two electrocardiograms, a term “mother electrocardiogram” and a term “fetal electrocardiogram” are used.

The electrocardiogram measurement apparatus 1 is mounted on the user S when being used, for example, as schematically illustrated in FIG. 1. For example, the electrocardiogram measurement apparatus 1 is mounted on the chest of the user S.

The electrocardiogram monitor device 6 has a function of displaying an electrocardiogram or the like on a display unit 61, based on measurement data transmitted from the electrocardiogram measurement apparatus 1. The electrocardiogram monitor device 6 may be a mobile terminal (for example, a tablet terminal or a smart phone) carried by the user S or may be a stationary type terminal. As schematically illustrated in FIG. 1, the electrocardiogram monitor device 6 is capable of wireless communication with the electrocardiogram measurement apparatus 1. However, the electrocardiogram monitor device 6 may be configured to communicate with the electrocardiogram measurement apparatus 1 via a wire. Hereinafter, it is assumed that the communication between the electrocardiogram monitor device 6 and the electrocardiogram measurement apparatus 1 is realized by wireless communication as an example. Meanwhile, the wireless communication may be based on a standard such as Bluetooth (registered trademark) or worldwide interoperability for microwave access (WiMAX).

Next, each embodiment of the electrocardiogram measurement apparatus suitable for being used for the electrocardiogram monitor system 100 will be described.

### Embodiment 1

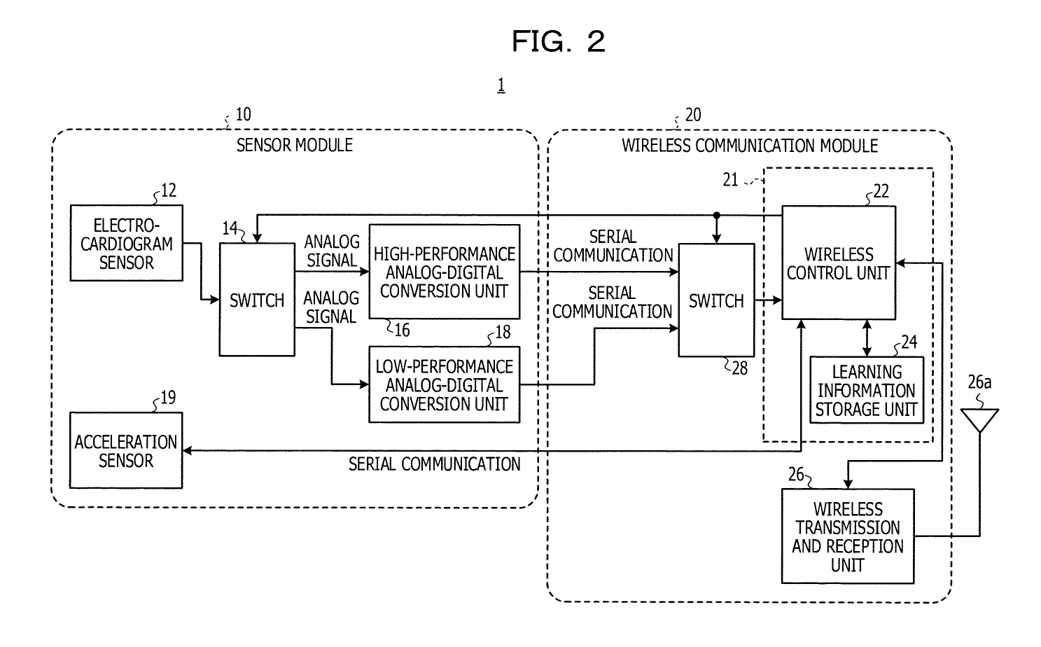

FIG. 2 is a diagram illustrating a configuration example of the electrocardiogram measurement apparatus 1 according to Embodiment 1.

The electrocardiogram measurement apparatus 1 has two or more measurement modes. In the example illustrated in FIG. 2, the electrocardiogram measurement apparatus 1 has two measurement modes of a low-accuracy measurement mode for measuring an electrocardiogram of a mother, and a high-accuracy measurement mode for measuring an electrocardiogram of a fetus in addition to the electrocardiogram of the mother. Details of the low-accuracy measurement mode and the high-accuracy measurement mode will be described below.

The electrocardiogram measurement apparatus 1 includes a sensor module 10 and a wireless communication module 20.

The sensor module 10 includes an electrocardiogram sensor 12 (an example of a biological sensor), a switch 14, and a high-performance analog-digital conversion unit 16 (hereinafter, referred to as a high-performance ADC 16) (an example of a first analog-digital conversion unit). In addition, the sensor module 10 further includes a low-performance analog-digital conversion unit 18 (hereinafter, referred to as a low-performance ADC 18) (an example of a second analog-digital conversion unit) and an acceleration sensor 19 (an example of a posture sensor).

The electrocardiogram sensor 12 generates an electrical signal (hereinafter, referred to as an “electrocardiogram signal”) corresponding to an electrocardiogram of the user S, and inputs the electrocardiogram signal to the switch 14. Meanwhile, the electrocardiogram signal output from the electrocardiogram sensor 12 is an analog signal (for example, −3.3 V to +3.3 V). The electrocardiogram sensor 12 is a contact-type sensor including an electrode to be attached to the chest of the user S, but may be a non-contact-type sensor. Then electrocardiogram sensor 12 may be a sensor to be attached to a portion other than the chest.

The switch 14 is switched between a first state and a second state in response to a switching signal (will be described below) input from a wireless communication module 20. In the first state of the switch 14, an electrocardiogram signal from the electrocardiogram sensor 12 is input to the high-performance ADC 16. Thereby, the high-accuracy mode is realized. In the second state of the switch 14, the electrocardiogram signal from the electrocardiogram sensor 12 is input to the low-performance ADC 18. Thereby, the low-accuracy mode is realized. By doing so, the electrocardiogram measurement apparatus 1 operates in the low-accuracy measurement mode or the high-accuracy measurement mode, according to a state of the switch 14 being switched in response to the switching signal (will be described below) input from the wireless communication module 20.

The high-performance ADC 16 functions in the high-accuracy measurement mode. The high-performance ADC 16 converts an analog electrocardiogram signal input from the electrocardiogram sensor 12 via the switch 14 into a digital electrocardiogram signal. The high-performance ADC 16 has at least one of a higher resolution and a higher sampling frequency than the low-performance ADC 18. The high-performance ADC 16 performs conversion processing at the resolution and the sampling frequency at which the fetal electrocardiogram can be measured. In Embodiment 1, the high-performance ADC 16 has both a higher resolution and a higher sampling frequency than the low-performance ADC 18 as an example. This is because a fetal electrocardiogram is significantly weaker than a mother electrocardiogram (an amplitude is small) and it is more advantageous that both the resolution and the sampling frequency of the fetal electrocardiogram are higher than those in a case where only the mother electrocardiogram is measured so as to accurately measure the fetal electrocardiogram. Specifically, the resolution and the sampling frequency of the high-performance ADC 16 are 24 bits and 200 Hz, respectively. In such a case, the fetal electrocardiogram can be accurately measured.

The low-performance ADC 18 functions in the low-accuracy measurement mode. The low-performance ADC 18 converts an analog signal input from the electrocardiogram sensor 12 via the switch 14 into a digital electrocardiogram signal. The low-performance ADC 18 performs conversion processing at a resolution and a sampling frequency in which the mother electrocardiogram can be measured. In Embodiment 1, the resolution and the sampling frequency of the low-performance ADC 18 are 16 bits and 100 Hz, respectively as an example.

The acceleration sensor 19 generates an electrical signal (hereinafter, referred to as an “acceleration signal”) (an example of posture information) corresponding to accelerations in three axial directions orthogonal to each other. The acceleration sensor 19 transmits the acceleration signal to the wireless communication module 20. In the example illustrated in FIG. 2, the acceleration sensor 19 transmits the acceleration signal to the wireless communication module 20 by serial communication.

Meanwhile, when the user S takes a predetermined stable posture, a gravitational acceleration appears in a direction of three axes. Since a method of mounting the electrocardiogram measurement apparatus 1 is the same every time, an angle relationship between the three axes in the gravitational direction does not change. Accordingly, each value (each component in the three axial directions) of the acceleration output by the acceleration sensor 19 is approximately the same value (that is, a value satisfying a certain allowable range) each time the user S reaches a predetermined stable posture.

The wireless communication module 20 generates measurement data, based on the acceleration signal from the acceleration sensor 19 and learning information (will be described below) stored therein, in accordance with the electrocardiogram sensor 12. The wireless communication module 20 transmits the generated measurement data to the outside (for example, the electrocardiogram monitor device 6 in FIG. 1).

The wireless communication module 20 includes a wireless control device 21, a wireless transmission and reception unit 26, and a switch 28.

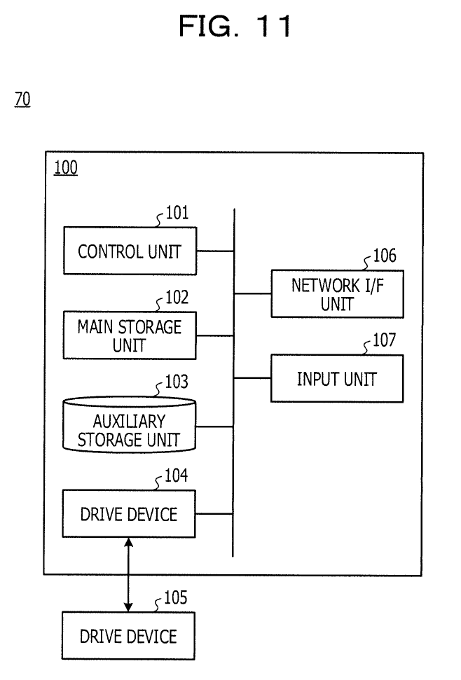

The wireless control device 21 is formed by, for example, a computer (see FIG. 11). The wireless control device 21 includes a wireless control unit 22 (an example of a processing unit) and a learning information storage unit 24.

The wireless control unit 22 determines a posture of the user S, based on the acceleration signal from the acceleration sensor 19. The wireless control unit 22 switches a measurement mode between the high-accuracy measurement mode and the low-accuracy measurement mode, based on the posture of the user S. Thereby, it is possible to change accuracy of the measurement data of the electrocardiogram sensor 12 depending on the posture of the user S.

The wireless control unit 22 preferably switches a measurement mode in a predetermined time zone between the high-accuracy measurement mode and the low-accuracy measurement mode, based on learning information on a posture of the user S in the predetermined time zone (or a plurality of time zones) in addition to the posture of the user S. Thereby, as will be described below, it is possible to determine a possibility (probability) that the user S will take a stable posture in the predetermined time zone. Accordingly, for example, by setting the measurement mode to the high-accuracy measurement mode only in a time zone in which there is a high possibility that the user S will take a stable posture, it is possible to achieve both an increase in opportunity to make a high-accuracy measurement and improvement in efficiency of power consumption, as will be described below. In Embodiment 1, it is assumed that the wireless control unit 22 switches the measurement mode, based on the posture of the user S and the learning information as an example.

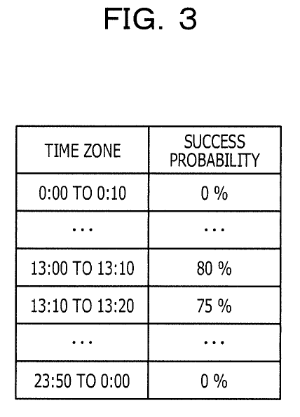

The wireless control unit 22 generates learning information on the posture of the user S for each time zone, based on the acceleration signal from the acceleration sensor 19, and stores the learning information in the learning information storage unit 24. The time zone is a time zone delimited every predetermined time in one day, and the predetermined time is random. For example, the time zone is a time zone delimited by the order of minutes and may be a time zone delimited every 10 minutes or every 3 minutes. Here, the acceleration signal from the acceleration sensor 19 represents a posture of the user S. The learning information may be data of the acceleration signal itself for each time zone, and may be information on the posture of the user S that can be derived from the acceleration signal, such as a type and an attribute of the posture of the user S for each time zone. In Embodiment 1, it assumed that the time zone is a time zone delimited every 10 minutes in one day and the learning information is a success probability for each time zone as an example as illustrated in FIG. 3. FIG. 3 illustrates a table chart representing an example of the success probability for each time zone. The success probability is a probability that a high-accuracy measurement is successfully made in a desired aspect. The desired aspect may include, for example, at least one success within 10 minutes, multiple successes within 10 minutes, multiple consecutive successes within 10 minutes, and the like. The high-accuracy measurement is a measurement in which the high-performance ADC 16 is used, and specifically, a measurement in the high-accuracy measurement mode. Meanwhile, the low-accuracy measurement is a measurement in which the low-performance ADC 18 is used, and specifically, a measurement in the low-accuracy measurement mode.

Whether or not a high-accuracy measurement is successful in a desired aspect may be determined based on feed-back information (for example, information indicating accuracy of the fetal electrocardiogram and the like) from the electrocardiogram monitor device 6. Alternatively, whether or not the high-accuracy measurement is successful in the desired aspect in a certain time zone may be determined based on the acceleration signal from the acceleration sensor 19 in the time zone. This can be used as a material for determining whether or not there is a posture of the user S which hinders success of the high-accuracy measurement in the time zone because the acceleration signal from the acceleration sensor 19 represents the posture of the user S. In Embodiment 1, the wireless control unit 22 determines whether or not the high-accuracy measurement is successful in the desired aspect, based on the acceleration signal from the acceleration sensor 19 as an example. For example, the wireless control unit 22 determines whether or not the acceleration signal from the acceleration sensor 19 satisfies a predetermined criterion when measurement starts in the high-accuracy measurement. In this case, the wireless control unit 22 may determine that the high-accuracy measurement is successful in a case where the acceleration signal from the acceleration sensor 19 satisfies the predetermined criterion when the measurement starts in the high-accuracy measurement. The predetermined criterion is a criterion for detecting the stable posture of the user S and may be set based on the acceleration signal when the user S is in the stable posture. Alternatively, the wireless control unit 22 may determine whether or not the acceleration signal from the acceleration sensor 19 satisfies the predetermined criterion during the measurement in the high-accuracy measurement instead of or in addition to when the measurement starts in the high-accuracy measurement. In this case, the wireless control unit 22 may determine that the high-accuracy measurement is successful during the measurement by the high-accuracy measurement in a case where the acceleration signal from the acceleration sensor 19 satisfies the predetermined criterion. Hereinafter, the learning information derived in this way by using the acceleration signals at a plurality of points of time during one measurement is referred to as “learning information under measurement” when it is pointed out in particular.

The wireless control unit 22 determines a measurement mode when the measurement starts. In Embodiment 1, the measurement mode is one of the high-accuracy measurement mode and the low-accuracy measurement mode as an example. The wireless control unit 22 generates a switching signal for switching between the high-accuracy measurement mode and the low-accuracy measurement mode, based on the acceleration signal from the acceleration sensor 19 and the learning information in the learning information storage unit 24. The wireless control unit 22 provides the generated switching signal to the switch 14 and the switch 28. For example, the wireless control unit 22 determines whether or not the acceleration signal satisfies a predetermined criterion when the measurement starts (for example, one point of time at which the measurement starts and/or a predetermined period immediately before the measurement starts). The predetermined criterion may be the same as described above. In addition, the wireless control unit 22 determines whether or not the success probability relating to the time zone to which a current time belongs is greater than or equal to a predetermined threshold value, based on the learning information. The predetermined threshold value corresponds to a comparatively high probability, and may be, for example, a value greater than or equal to 70[%]. The predetermined threshold value may be set by the user S. In a case where the acceleration signal satisfies the predetermined criterion when the measurement starts and the success probability relating to the time zone to which the current time belongs is greater than or equal to the predetermined threshold value, the wireless control unit 22 generates the switching signal such that measurement in the high-accuracy measurement mode is realized. Meanwhile, in a case where the acceleration signal does not satisfy the predetermined criterion when the measurement starts or in a case where the success probability relating to the time zone to which the current time belongs is not greater than or equal to the predetermined threshold value, the wireless control unit 22 generates the switching signal such that the measurement in the low-accuracy measurement mode is realized. Each of the switch 14 and the switch 28 may form a state (a first state or a second state) realizing either one of the high-accuracy measurement mode and the low-accuracy measurement mode in a normal state. In this case, the switching signal may be provided to the switch 14 and the switch 28 only when a state other the normal state is formed. In the example illustrated in FIG. 2, two switches 14 and 28 are provided, but either one of the two switches 14 and 28 may be omitted.

The wireless control unit 22 acquires an electrocardiogram signal from the electrocardiogram sensor 12 input via the switch 28. In the high-accuracy measurement mode, the wireless control unit 22 acquires the electrocardiogram signal converted by the high-performance ADC 16. In the low-accuracy measurement mode, the wireless control unit 22 acquires the electrocardiogram signal converted by the low-performance ADC 18. The wireless control unit 22 acquires measurement data of the electrocardiogram sensor 12 to the wireless transmission and reception unit 26, based on the acquired electrocardiogram signal.

The learning information storage unit 24 stores learning information. The learning information storage unit 24 may be, for example, a nonvolatile memory (for example, a flash memory, a hard disk drive, or the like) that can be rewritten.

The wireless transmission and reception unit 26 generates a transmission signal for transmitting the measurement data received from the wireless control unit 22. The wireless transmission and reception unit 26 transmits the generated transmission signal to the electrocardiogram monitor device 6 via the antenna 26a.

The switch 28 is switched between the first state and the second state in response to the switching signal input from the wireless control unit 22. The first state of the switch 28 is formed in the high-accuracy measurement mode. In the first state of the switch 28, the electrocardiogram signal from the high-performance ADC 16 is provided to the wireless control unit 22. Thereby, the wireless transmission and reception unit 26 can generate the transmission signal, based on the electrocardiogram signal obtained from the high-performance ADC 16. The second state of the switch 28 is formed during the low-accuracy measurement mode. In the second state of the switch 28, the electrocardiogram signal from the low-performance ADC 18 is provided to the wireless control unit 22. Thereby, the wireless transmission and reception unit 26 can generate the transmission signal, based on the electrocardiogram signal obtained from the low-performance ADC 18. By doing so, the wireless communication module 20 transmits measurement data with different accuracy depending on the measurement mode of the electrocardiogram measurement apparatus 1 to the electrocardiogram monitor device 6.

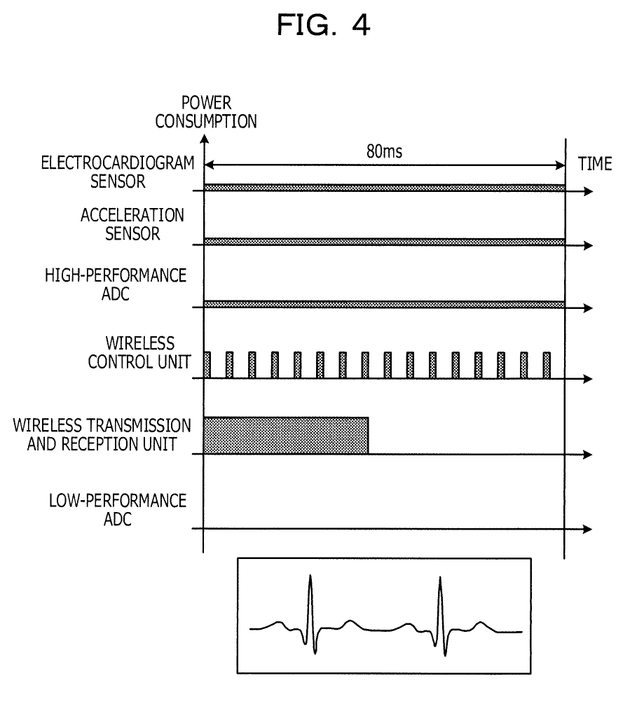

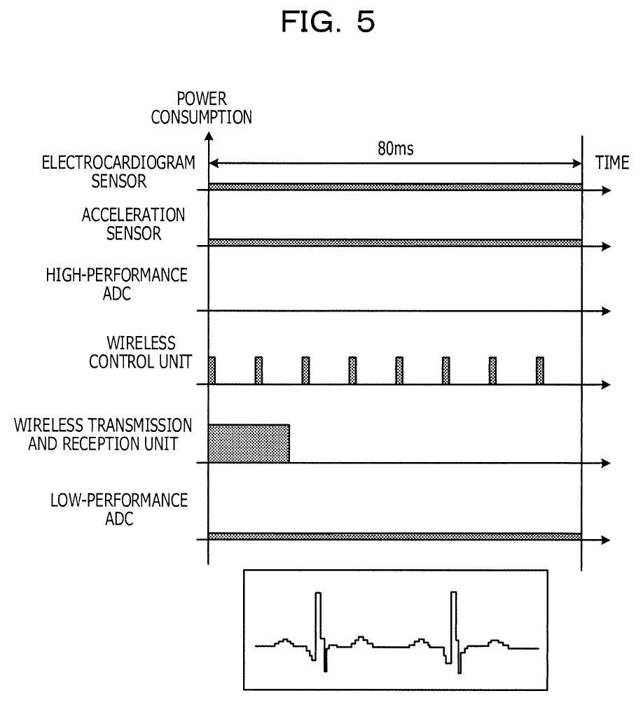

FIG. 4 is a diagram schematically illustrating a time series of power consumption of the electrocardiogram sensor 12 and the like in the high-accuracy measurement mode. FIG. 5 is a diagram schematically illustrating the time series of the power consumption of the electrocardiogram sensor 12 and the like in the low-accuracy measurement mode. Specifically, in FIGS. 4 and 5, the time series of each power consumption of the electrocardiogram sensor 12, the acceleration sensor 19, the high-performance ADC 16, the wireless control unit 22, the wireless transmission and reception unit 26, and the low-performance ADC 18 are schematically illustrated in order from the top. In FIG. 4 and FIG. 5, measurements for a measurement period of 80 ms are illustrated. In addition, in FIG. 4 and FIG. 5, an image diagram of a waveform displayed on the display unit 61 of the electrocardiogram monitor device 6 is illustrated on the lowermost side. The power consumed in the wireless transmission and reception unit 26 illustrated in FIG. 4 is power consumed when the measurement data obtained in the previous (another measurement period of 80 ms immediately before the measurement period illustrated in FIG. 4) high-accuracy measurement is processed, and the power consumed in the wireless transmission and reception unit 26 illustrated in FIG. 5 is power consumed when the measurement data obtained in the previous low-accuracy measurement is processed.

In the example illustrated in FIG. 4, the high-performance ADC 16 converts the electrocardiogram signal into a digital signal with 16 samples×24 bits (3 bytes) during the measurement period of 80 ms. Meanwhile, in the example illustrated in FIG. 5, the low-performance ADC 18 converts the electrocardiogram signal into a digital signal with 8 samples×16 bits (2 bits) during the measurement period of 80 ms. As a result, in the wireless control unit 22 in the high-accuracy measurement mode, the number of acquisitions of the electrocardiogram signal is 16 for 16 samples, which is twice the number of acquisitions in the low-accuracy measurement mode, and the power consumption is also approximately twice. In addition, in the high-accuracy measurement mode, the wireless transmission and reception unit 26 transmits measurement data of three packets, based on the electrocardiogram signal from the high-performance ADC 16. In contrast, in the low-accuracy measurement mode, the wireless transmission and reception unit 26 transmits the measurement data of one packet, based on the electrocardiogram signal from the low-performance ADC 18. As a result, in the radio transmission and reception unit 26 in the high-accuracy measurement mode, the power consumption also increases as much as the number of packets to be transmitted, compared with the power consumption in the low-accuracy measurement mode. In the examples illustrated in FIGS. 4 and 5, the transmission signal to be transmitted to the electrocardiogram monitor device 6 includes the measurement data of the acceleration sensor 19 in addition to the measurement data of the electrocardiogram sensor 12. The measurement data of the acceleration sensor 19 is transmitted as one packet together with the header.

As described above, according to Embodiment 1, a measurement mode is switched between the high-accuracy measurement mode and the low-accuracy measurement mode depending on a posture of the user S, and thus, it is possible to change the accuracy of the measurement data of the electrocardiogram sensor 12 depending on the posture of the user S.

Here, since an electrocardiogram of a fetus is a very small signal, there is a possibility that the electrocardiogram of the fetus may not be acquired with high accuracy even in the high-accuracy measurement mode, in a case where noise is included in the electrocardiogram signal acquired by the wireless control unit 22, such as when the user S is not in a stable posture. That is, even in the high-accuracy measurement mode, there is a possibility that a high-accuracy measurement of the electrocardiogram of the fetus may not be performed depending on the posture of the user S.

Accordingly, when the user S is not in a stable posture, it is desirable to perform measurement in the low-accuracy measurement mode with relatively low power consumption without performing the measurement in the high-accuracy measurement mode with relatively high power consumption from the viewpoint of power consumption.

In this regard, according to Embodiment 1, as described above, the posture of the user S is determined based on the acceleration signal when the measurement starts, and thereby, only when the user S is in a stable posture at the start of starting the measurement, measurement in the high-accuracy measurement mode can be performed. Specifically, when the user S is in a stable posture, a high-accuracy measurement can be realized, and when the user S is not in a stable posture, a low-accuracy measurement can be performed. Thereby, it is possible to improve efficiency of power consumption of the electrocardiogram measurement apparatus 1 and to improve high accuracy of the measurement data of the electrocardiogram sensor 12, depending on the posture of the user S.

In addition, according to Embodiment 1, as described above, a measurement mode is switched between a high-accuracy measurement mode and a low-accuracy measurement mode, based on learning information on a posture of the user S for each time zone. Thereby, high accuracy measurement can be attempted in a time zone in which there is a high possibility that the user S will be in a stable posture. Thereby, it is possible to achieve both an increase in opportunities to perform a high-accuracy measurement and the improvement in efficiency of power consumption.

Here, even if the user S is in a stable posture at the time of starting the measurement, the user S may not be in a stable posture during measurement after the measurement starts. Also in such a case, there is a possibility that high-accuracy measurement of the fetal electrocardiogram may not be performed, and thus, it is desirable that the measurement is performed in the low-accuracy measurement mode in which power consumption is relatively low, without performing the measurement in the high-accuracy measurement mode in which power consumption is relatively high, from the viewpoint of power consumption.

In this regard, in the configuration in which learning information is used during above-described measurement, the possibility that a stable posture of a user S is continuously maintained during the high-accuracy measurement is determined based on the learning information during measurement, and thereby, it is possible to reduce inconvenience in a case where the user S is not in a stable posture during the high-accuracy measurement after the high-accuracy measurement starts. That is, as described above, since the in-measurement learning information on the posture of the user S in the same time zone in the past is used, it is possible to accurately determine the possibility that a stable posture of the user S will be continuously maintained during the measurement in the same time zone. As a result, in a case where there is a high possibility that the user S is not in a stable posture during the high-accuracy measurement after the high-accuracy measurement starts, a situation that the high-accuracy measurement starts is reduced, and high-accuracy measurement can be made only in the time zone in which there is a possibility that the user S is continuously in the stable posture. Thereby, it is possible to further achieve both the increase in opportunity to make the high-accuracy measurement with a high accuracy and the improvement in efficiency of power consumption. A configuration using the in-measurement learning information is suitable for a measurement application in which one measurement time is relatively long.

In Embodiment 1, the high-accuracy measurement mode and the low-accuracy measurement mode are switched to each other but can be switched to other aspects. For example, instead of being measured in the low-accuracy measurement mode, measurement may not be made (that is, the measurement may be prohibited). Hereinafter, such a modification example will be referred to as a “first modification example”. The first modification is suitable in a case where an electrocardiogram desired by the user S is not a mother electrocardiogram but a fetal electrocardiogram (or in a case of the mother electrocardiogram and the fetal electrocardiogram). A switching aspect according to the first modification example and a switching aspect according to the above-described Embodiment 1 may be selectable by the user S, for example, by a setting change and the like. In the first modification example, prohibition of measurement may be realized by deactivating the electrocardiogram sensor 12, deactivating the high-performance analog-digital conversion unit 16 and the low-performance analog-digital conversion unit 18, deactivating the wireless communication module 20 or the like.

In addition, in Embodiment 1, a success probability for each time zone may be calculated irrespective of the day of the week, the season or the like, and may be calculated according to the day of the week, the season, or the like. Hereinafter, such a modification example will be referred to as a “second modification example”. For example, the success probability for each time zone may separately include the success probability for each time zone on a weekday and the success probability for each time zone on a holiday. This is because a life pattern of the user S may be different between the weekday and the holiday. From the same point of view, the success probability for each time zone may be calculated separately for each season, or may be calculated separately for each climate.

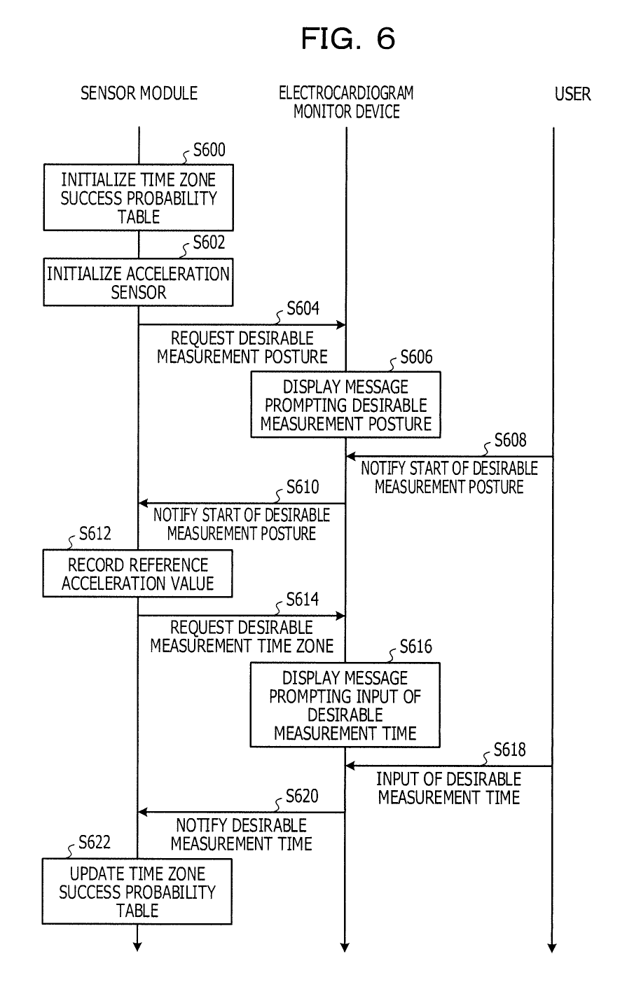

Next, an operation example of the electrocardiogram monitor system 100 will be described with reference to FIGS. 6 to 10.

FIG. 6 is a sequence diagram illustrating an example of initial setting processing of the electrocardiogram monitor system 100. FIG. 6 illustrates a relationship between the electrocardiogram measurement apparatus 1, the electrocardiogram monitor device 6, and the user S at the time of initial setting. The sequence illustrated in FIG. 6 may be performed for example, when the electrocardiogram measurement apparatus 1 and the electrocardiogram monitor device 6 are powered on by the user S and a connection between the electrocardiogram measurement apparatus 1 and the electrocardiogram monitor device 6 is established. In addition, the sequence illustrated in FIG. 6 may be performed, for example, when the initialization setting completion flag is “0 (FALSE)”. An initial value of the initialization setting completion flag is “0”. The initial setting completion flag may be able to be initialized by the user S.

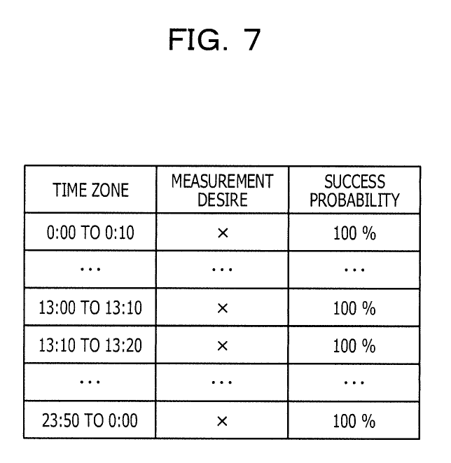

The wireless control unit 22 of the electrocardiogram measurement apparatus 1 initializes a table (hereinafter, referred to as a “time zone success probability table”) representing a desired time zone and success probability (step S600). FIG. 7 illustrates an example of the time zone success probability table in an initial state. FIG. 7 illustrates information indicating presence or absence of measurement desire and success probability for each time zone. For the information indicating the presence or absence of the measurement desire, ◯ (not illustrated) represents “with desire”, and x represents “no desire”. An initial value of the information indicating presence or absence of the measurement desire is, for example, “no desire”. The initial value of the success probability is, for example, “100”.

Next, the wireless control unit 22 initializes the acceleration sensor 19 (step S602). Next, the wireless control unit 22 requests the electrocardiogram monitor device 6 to acquire the desirable posture (hereinafter, referred to as a “measurement desirable posture”) of the user S regarding a posture taken by the user S during measurement (step S604).

The electrocardiogram monitor device 6 outputs a message prompting the user S to take a desirable measurement posture in a state where the electrocardiogram measurement apparatus 1 is mounted and to press a predetermined button of the electrocardiogram monitor device 6 in the desirable measurement posture, to the display unit 61 (step S606). Other outputs (including a sound output, flickering of a light source, and the like) may be used instead of outputting a message to the display unit 61. The user S takes a desirable measurement posture in a state where the electrocardiogram measurement apparatus 1 is mounted, and presses a predetermined button (not illustrated) of the electrocardiogram monitor device 6 in the desirable measurement posture. The desirable measurement posture is basically a stable posture, and, for example, a seated posture, a posture in which a user lies on a bed, or the like. Instead of pressing the predetermined button, other operations (including a voice input, a gesture input, and the like) may be used. If the predetermined button of the electrocardiogram monitor device 6 is pressed (step S608), the electrocardiogram monitor device 6 transmits information indicating that the measurement desired posture is being taken to the electrocardiogram measurement apparatus 1 (step S610).

If receiving the information indicating that the desirable measurement posture is being taken, the wireless control unit 22 acquires the acceleration signal from the acceleration sensor 19 and stores each value (aₓ₀, a_{y0}, a_{z0}) of accelerations in three axial directions in the learning information storage unit 24 (step S612). Each value (aₓ₀, a_{y0}, a_{z0}) of accelerations in three axial directions may be an average value during a certain period or may be a representative value during a certain period. Each value (aₓ₀, a_{y0}, a_{z0}) of the accelerations in the three axial directions functions as a reference value for determining whether or not a posture of the user S is a desirable measurement posture, and is hereinafter referred to as a “reference acceleration value”. Next, the wireless control unit 22 requests the electrocardiogram monitor device 6 to acquire a time zone desired to be measured (step S614).

The electrocardiogram monitor device 6 outputs a message for prompting the user S to input the time zone desired to be measured to the display unit 61 (step S616). The user S inputs the time zone desired to be measured from an operation unit (not illustrated) of the electrocardiogram monitor device 6. At this time, it is desirable that the user S inputs a time zone (for example, a time zone of a lunch break) in which there is a high possibility that a stable posture will be positively taken, or a time zone (for example, a time zone immediately before going to bed) in which there is a high possibility that a stable posture is naturally taken, as a time zone in which measurement is desired. Thereby, it is possible to make measurement in a time zone in which there is a high probability that the user S is in a stable posture. If the time zone desired to be measured is input (step S618), the electrocardiogram monitor device 6 transmits the time zone desired to be measured to the electrocardiogram measurement apparatus 1 (step S620).

If receiving the time zone desired to be measured, the wireless control unit 22 updates the time zone success probability table (see FIG. 7) such that the time zone desired to be measured becomes “with desire”. If the time zone success probability table is updated based on the time zone desired to be measured, the wireless control unit 22 sets a value of an initial setting completion flag to “1 (TRUE)”. Hereinafter, the time zone which becomes “with desire” in the time zone success probability table is referred to as a “desirable measurement time zone”.

In the example illustrated in FIG. 6, the user S inputs a time zone desired to be measured, but the user S may input time desired to be measured. In this case, a time zone to which the desired time belongs may be treated as the desirable measurement time zone.

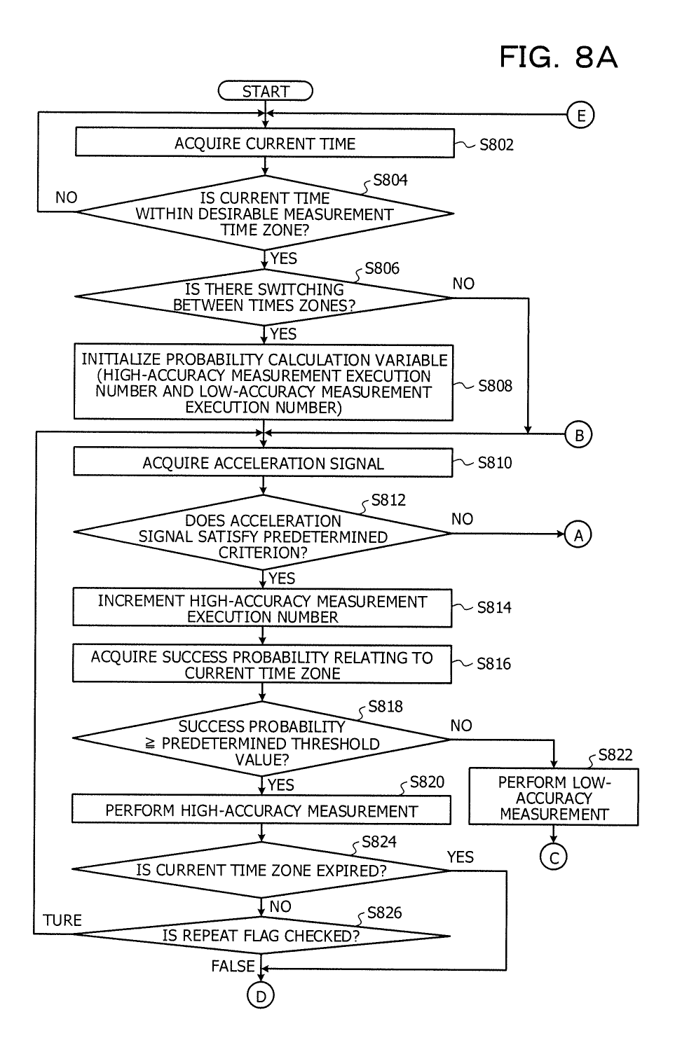

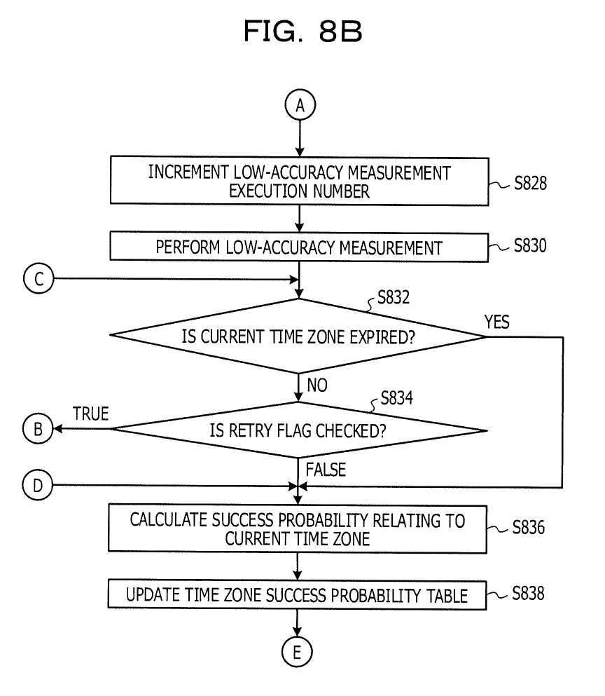

FIGS. 8A and 8B are a flowchart illustrating an example of measurement processing performed by the electrocardiogram measurement apparatus 1. The processing illustrated in FIGS. 8A and 8B may be performed, for example, when the electrocardiogram measurement apparatus 1 and the electrocardiogram monitor device 6 are power on by the user S, a connection between the electrocardiogram measurement apparatus 1 and the electrocardiogram monitor device 6 is established, and the initial setting completion flag is set to “1”. Alternatively, the processing illustrated in FIGS. 8A and 8B may be performed when a measurement start instruction from the user S is input via the electrocardiogram monitor device 6.

In the processing illustrated in FIGS. 8A and 8B, a retry flag and a repeat flag are used as an example. The retry flag and the repeat flag are set in advance from the following viewpoint: In a case where a user wants to measure the entire time zone regardless of measurement accuracy in one desirable measurement time zone, the retry flag is set to TRUE and the repeat flag is set to TRUE. In a case where the high-accuracy measurement may be able to be performed at least once in the one desirable measurement time zone, the retry flag is set to TRUE and the repeat flag is set to FALSE. If the user wants to perform a high-accuracy measurement as continuously as possible from the beginning in one desirable measurement time zone (that is, in a case where the user wants to end the measurement at a point of time in a case of a low-accuracy measurement even once), the retry flag is set to FALSE and the repeat flag is set to TRUE. In a case where the user wants to perform measurement only once regardless of whether the measurement is a low-accuracy measurement or a high-accuracy measurement in one desirable measurement time zone, the retry flag is set to FALSE, and the repeat flag is set to FALSE. In this way, setting for the retry flag and the repeat flag depends on a measurement usage and the like. The setting for the retry flag and the repeat flag may be able to be changed by the user S.

In step S802, the wireless control unit 22 acquires a current time. The current time can be acquired based on, for example, a timepiece or a timer (both are not illustrated) that are embedded in the electrocardiogram measurement apparatus 1.

In step S804, the wireless control unit 22 determines whether or not a time zone to which the current time belongs is a desirable measurement time zone with reference to the time zone success probability table (see FIG. 7). In a case where it is determined that the time zone to which the current time belongs is the desirable measurement time zone, the processing illustrated in FIGS. 8A and 8B proceeds to step S806, and in the other cases, the processing illustrated in FIGS. 8A and 8B starts again from step S802 after a predetermined time passes (for example, after one minute). Hereinafter, the time zone to which the current time belongs is referred to as a “current time zone”.

In step S806, the wireless control unit 22 determines whether or not there is switching between time zones. The switching between time zones is switching between a plurality of time zones illustrated in FIG. 7. In a case where there is the switching of time zone, the processing illustrated in FIGS. 8A and 8B proceeds to step S810 via step S808, and in the other cases, the processing illustrated in FIGS. 8A and 8B skips step S808 and proceeds to step S810.

In step S808, the wireless control unit 22 initializes a probability calculation variable to zero. The probability calculation variable includes a high-accuracy measurement execution number and a low-accuracy measurement execution number. The high-accuracy measurement execution number represents the number of executions of the high-accuracy measurement in the current time zone, and the low-accuracy measurement execution number represents the number of executions of the high-accuracy measurement in the current time zone.

In step S810, the wireless control unit 22 acquires an acceleration signal from the acceleration sensor 19.

In step S812, the wireless control unit 22 determines whether the acquired acceleration signal satisfies a predetermined criterion. That is, the wireless control unit 22 determines whether or not a posture of the user S is the desirable measurement posture (stable posture). The predetermined criterion is set based on a reference acceleration value. Thereby, it is possible to accurately determine whether or not the posture of the user S is in the desirable measurement posture. For example, the wireless control unit 22 determines whether or not each value in the three axial directions of the acquired acceleration signal is within an error range of the reference acceleration value. In a case where the posture of the user S is the desirable measurement posture, the processing illustrated in FIGS. 8A and 8B proceeds to step S814, and in the other cases, the processing illustrated in FIGS. 8A and 8B proceeds to step S828.

In step S814, the wireless control unit 22 increments a value of the high-accuracy measurement execution number only by “1”.

In step S816, the wireless control unit 22 acquires a success probability relating to the current time zone with reference to the time zone success probability table (see FIG. 7).

In step S818, the wireless control unit 22 determines whether or not the success probability relating to the current time zone is greater than or equal to a predetermined threshold value. In a case where the success probability is greater than or equal to the predetermined threshold value, the processing illustrated in FIGS. 8A and 8B proceeds to step S820, and in the other cases, the processing illustrated in FIGS. 8A and 8B proceeds to step S822.

In step S820, the wireless control unit 22 performs a high-accuracy measurement (measurement in a high-accuracy measurement mode). The high-accuracy measurement may be continuously performed for a predetermined period (for example, one minute). If the high-accuracy measurement (high-accuracy measurement for one time) for the predetermined period is completed, the processing illustrated in FIGS. 8A and 8B proceeds to step S824.

In step S822, the wireless control unit 22 performs a low-accuracy measurement (measurement in a low-accuracy measurement mode). The low-accuracy measurement may be continuously performed for a predetermined period (for example, one minute). If the low-accuracy measurement (low-accuracy measurement for one time) for the predetermined period is completed, the processing illustrated in FIGS. 8A and 8B proceeds to step S824.

In step S824, the wireless control unit 22 determines whether or not the current time zone is expired. For example, when the current time zone is “13:00-13:10”, the current time zone is expired at 13:10. In a case where the current time zone is expired, the processing illustrated in FIGS. 8A and 86 proceeds to step S836, and in the other cases, the processing illustrated in FIGS. 8A and 8B proceeds to step S826.

In step S826, the wireless control unit 22 checks the repeat flag. In a case where the repeat flag is “TRUE”, the processing illustrated in FIGS. 8A and 8B returns to step S810, and a new measurement is repeated. Meanwhile, in a case where the repeat flag is “FALSE”, the processing illustrated in FIGS. 8A and 8B proceeds to step S836.

In step S828, the wireless control unit 22 increments a value of the low-accuracy measurement execution number only “1”.

In step S830, the wireless control unit 22 performs a low-accuracy measurement (measurement in the low-accuracy measurement mode). The low-accuracy measurement may be continuously performed for a predetermined period (for example, one minute). If the low-accuracy measurement (low-accuracy measurement for one time) for the predetermined period is completed, the processing illustrated in FIGS. 8A and 8B proceeds to step S832.

In step S832, the wireless control unit 22 determines whether or not the current time zone is expired. In a case where the current time zone is expired, the processing illustrated in FIGS. 8A and 8B proceeds to step S836, and in the other cases, the processing illustrated in FIGS. 8A and 8B proceeds to step S834.

In step S834, the wireless control unit 22 checks the retry flag. In a case where the retry flag is “TRUE”, the processing illustrated in FIGS. 8A and 8B returns to step S810, and a new high-accuracy measurement is attempted. Meanwhile, in a case where the retry flag is “FALSE”, the processing proceeds to step S836.

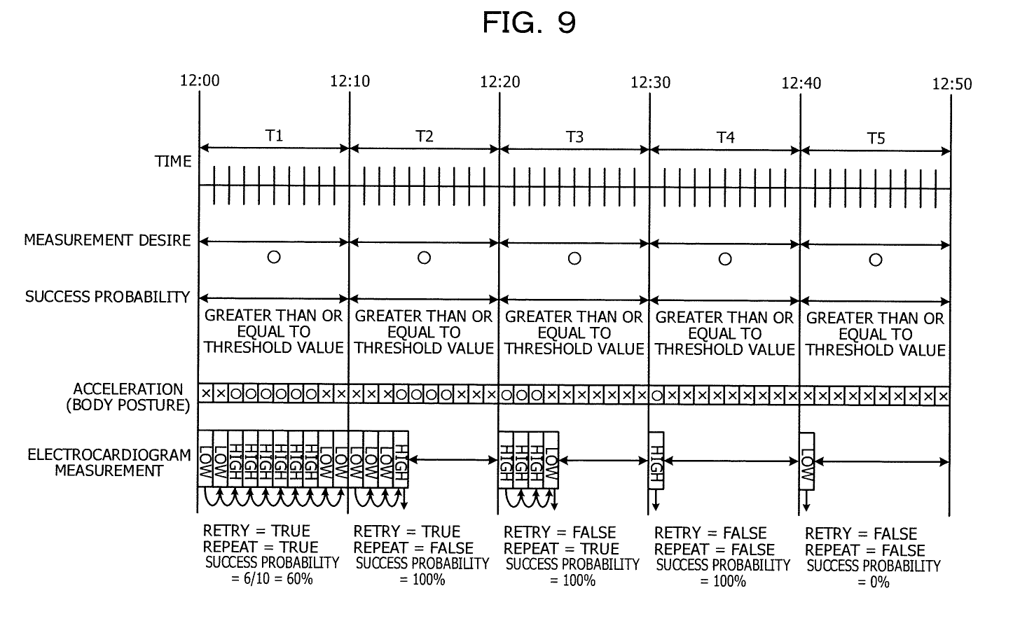

In step S836, the wireless control unit 22 calculates a success probability in the current time zone, based on each value of the high-accuracy measurement execution time and the low-accuracy measurement execution time, and each state of the repeat flag and the retry flag. FIG. 9 is an explanatory diagram of a method of calculating the success probability in step S836. In FIG. 9, five time zones T1 to T5 are denoted every 10 minutes from 12:00 to 12:50. In FIG. 9, it is assumed that one measurement (interval) takes 1 minute, all the time zones T1 to T5 are the desirable measurement time zones, and the success probability relating to all the time zones T1 to T5 is greater than or equal to a predetermined threshold value. In FIG. 9, each section of the acceleration is classified corresponding to each measurement period every minute, “◯” in the section means that an acceleration signal in the measurement period satisfies a predetermined criterion, and “x” means that the acceleration signal in the measurement period does not satisfy the predetermined criterion. In addition, in FIG. 9, each section of the electrocardiogram measurement is divided corresponding to each measurement of one time, “high” in the section means that the high-accuracy measurement is performed, and “low” in the section means that the low-accuracy measurement is performed.

In a case where the retry flag is TRUE and the repeat flag is TRUE, a success probability Ps in the current time zone may be as follows.

Ps=N1/(N1+N2)

Here, N1 is a value of the high-accuracy measurement execution number, and N2 is a value of the low-accuracy measurement execution number. In FIG. 9, the retry flag is TRUE and the repeat flag is TRUE in a time zone T1 between of 12:00 and 12:10, 10 measurements are performed, and N1=6 and N2=4. In this case, Ps=60 (=6/10×100) [%].

Meanwhile, in a case where both the retry flag and the repeat flag are not TRUE (that is, in a case where the retry flag is not TRUE or the repeat flag is not TRUE), the success probability Ps in the current time zone depends on the value of the high-accuracy measurement execution number, and may be as follows.

Ps=100 (N1≥1)

Ps=0 (N1=0)

In FIG. 9, in a time zone T2 between 12:10 and 12:20, the retry flag is TRUE and the repeat flag is FALSE, and the high-accuracy measurement can be realized in a fourth measurement. In this case, Ps becomes 100[%]. In addition, in a time zone T3 between 12:20 and 12:30, the retry flag is FALSE and the repeat flag is TRUE, and the high-accuracy measurement can be consecutively performed from the first measurement to the third measurement. In this case, Ps becomes 100[%]. In addition, in a time zone T4 between 12:30 and 12:40, the retry flag is FALSE and the repeat flag is FALSE, and the first measurement is the high-accuracy measurement. In this case, Ps becomes 100[%]. In addition, in a time zone T5 between 12:40 and 12:50, the retry flag is FALSE and the repeat flag is FALSE, and the first measurement is the low-accuracy measurement. In this case Ps becomes 0[%].

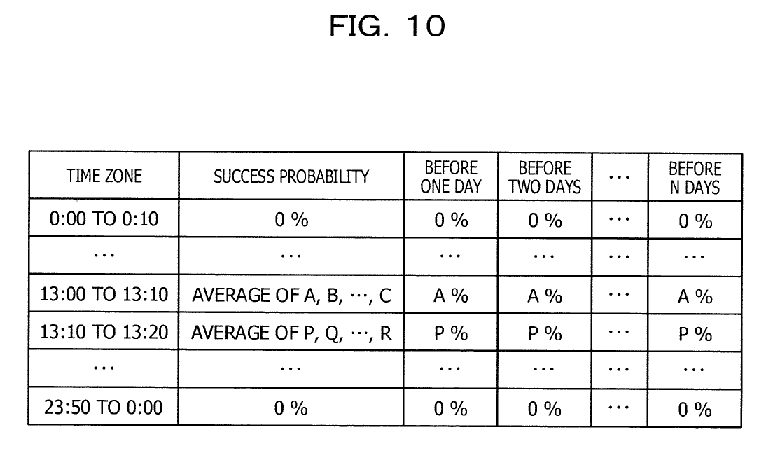

In step S838, the wireless control unit 22 updates the time zone success probability table, based on the success probability Ps, which is calculated in step S836, in the current time zone. For example, the wireless control unit 22 may overwrite the success probability in the same time zone in the time zone success probability table with the success probability Ps in the current time zone calculated in step S836. Alternatively, the wireless control unit 22 may update the success probability in the same time zone in the time zone success probability table by averaging the success probability Ps in the current time zone calculated in step S836 and each success probability in the same time zone corresponding to a predetermined number of recent days. FIG. 10 illustrates a table in which the success probabilities for N days is stored. In this case, the success probability for each time zone is calculated by averaging the success probabilities of N days for each time zone. For example, the success probability relating to the time zone between 13:00 and 13:10 is calculated by averaging the success probabilities (A, B, . . . , C) for N days in the same time zone. If the processing of step S838 ends, the processing illustrated in FIGS. 8A and 8B returns to step S802.

In the example illustrated in FIGS. 8A and 8B, the repeat flag and the retry flag are used so as to be able to flexibly cope with a change or the like of a measurement usage, but any one or both of the flags may be omitted (hereinafter, the modification example is referred to as a “third modification example”). For example, in a case where the retry flag may be fixed to TRUE and the repeat flag may be fixed to TRUE, the determinations in step S826 and step S834 may be omitted.

In the example illustrated in FIGS. 8A and 8B, whether or not there is a possibility that a stable posture of the user S is maintained is determined based on learning information in step S818, but the learning information may not be used (hereafter, the modification example is referred to as a “fourth modification example”). In this case, the step S808, the step S814, the step S816, the step S818, the step S822, the step S828, the step S836, and the step S838 may be omitted in FIGS. 8A and 8B. In FIGS. 8A and 8B, in a case where the result of the determination in step S812 is “YES”, the processing illustrated in FIGS. 8A and 8B may proceed to step S820. In the fourth modification example, actually, the measurement mode is switched between the high-accuracy measurement mode and the high-accuracy measurement mode, based on only the acceleration signal when the measurement starts. The fourth modification example is suitable in a case where two kinds of biological signals (that is, the mother electrocardiogram and the fetal electrocardiogram) are measured by a common biological sensor as in Embodiment 1 described above and one measurement period is relatively short.

In addition, in the example illustrated in FIGS. 8A and 8B, whether or not the time zone to which the current time belongs is the desirable measurement time zone is determined in Step S804, but the determination may be omitted (hereinafter, the modification example is referred to as a fifth modification example). In the fifth modification example, steps S614 to S622 may be omitted in the initial setting processing illustrated in FIG. 6.

In addition, in the example illustrated in FIGS. 8A and 8B, the determination in step S818 is performed only when the measurement starts but may be continuously performed during the measurement after the measurement starts (hereinafter, the modification example is referred to as a “sixth modification example”). That is, the above-described in-measurement learning information may be generated. In the sixth modification example, step S814 is omitted, and instead thereof, the determination of step S812 is repeated step S820 may be performed during the measurement in step S820. Then, in a case where the number of times or a ratio at which the determination result of step S812 is “YES” during one measurement made in step S820 is greater than or equal to the predetermined threshold value, the value of the high-accuracy measurement execution number may be incremented only by “1”.

The third to sixth modification examples can be combined in a random aspect.

FIG. 11 is diagram illustrating an example of a hardware configuration of the wireless control device 21 of the electrocardiogram monitor device 6. In the example illustrated in FIG. 11, the wireless control device 21 includes a control unit 101, a main storage unit 102, an auxiliary storage unit 103, a drive device 104, a network I/F unit 106, and an input unit 107.

The control unit 101 is an arithmetic device that executes a program stored in the main storage unit 102 or the auxiliary storage unit 103, receives data from the input unit 107 or the storage device, an arithmetic operation and processing for the data are performed, and thereafter, the data is output to the storage device or the like.

The main storage unit 102 is a read only memory (ROM), a random access memory (RAM), or the like. The main storage unit 102 is a storage device that stores or temporarily retains an operating system (OS) that is a basic software executed by the control unit 101, a program such as an application software, and data.

The auxiliary storage unit 103 is a storage device that is a flash memory, a hard disk drive (HDD), or the like and stores data relating to application software. The auxiliary storage unit 103 may form the learning information storage unit 24 described above.

The drive device 104 reads a program from a recording medium 105, for example, a flexible disk, and installs the program in the storage device.

The recording medium 105 stores a program. The program stored in the recording medium 105 is installed in the wireless control device 21 via the drive device 104. The installed predetermined program can be executed by the wireless control device 21.

The network I/F unit 106 is an interface between a peripheral device (for example, a sensor module 10 or the wireless transmission and reception unit 26) having a communication function via a network constructed by a data transmission line such as a wired and/or a wireless line, and the wireless control device 21.

The input unit 107 includes a keyboard, a mouse, a touch pad, and the like, which include a cursor key, numeric input and various function keys, and the like.

In the example illustrated in FIG. 11, the above-described various kinds of processing of the wireless control unit 22 can be realized by executing a program in the wireless control device 21. In addition, it is also possible to realize various kinds of processing of the wireless control unit 22 described above by recording the program in the recording medium 105 and making the wireless control device 21 read the program stored in the recording medium 105. The recording medium 105 can use various kinds of recording media. For example, the recording medium 105 may be a recording medium such as a compact disc (CD)-ROM, a flexible disk, or a magnetooptical disk for recording information optically, electrically or magnetically, a semiconductor memory such a ROM or a flash memory for electrically recording information, or the like. The recording medium 105 does not include a carrier wave.

### Embodiment 2

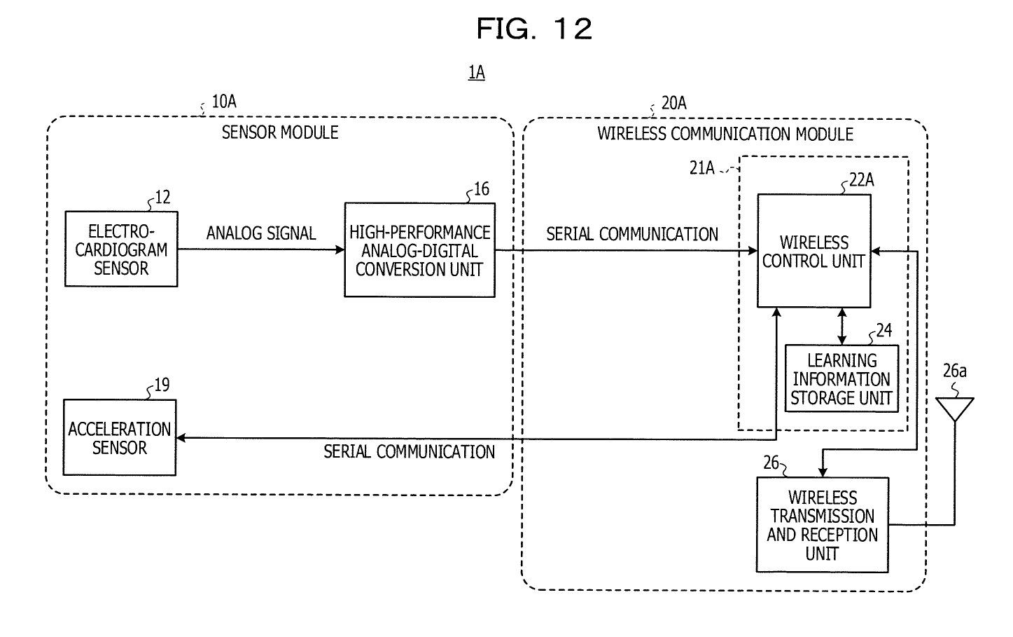

FIG. 12 is a diagram illustrating a configuration example of an electrocardiogram measurement apparatus 1A (an example of a measurement apparatus) according to Embodiment 2. In Embodiment 2 which will be described below, the same configuration elements as those described in Embodiment 1 described above are denoted by the same reference numerals or symbols as in FIG. 12 and description thereof will be omitted.

In Embodiment 1 described above, the accuracy of the measurement data of the electrocardiogram sensor 12 is changed by switching between the high-performance ADC 16 and the low-performance ADC 18, but in the following Embodiment 2, the accuracy of the measurement data of the electrocardiogram sensor 12 is changed in an aspect different from the above-described Embodiment 1. Although description will be specifically made hereinafter, the accuracy of the measurement data of the electrocardiogram sensor 12 is changed by a wireless communication module 20A in Embodiment 2.

The electrocardiogram measurement apparatus 1A includes a sensor module 10A and a wireless communication module 20A.

The sensor module 10A is different from the sensor module 10 according to Embodiment 1 described above in that the switch 14 and the low-performance ADC 18 are omitted. An analog electrocardiogram signal from the electrocardiogram sensor 12 is input to the high-performance ADC 16. The high-performance ADC 16 converts the analog electrocardiogram signal into a digital electrocardiogram signal. The high-performance ADC 16 outputs the digital electrocardiogram signal to a wireless control unit 22A through a serial communication.

The wireless communication module 20A is different from the wireless communication module 20 according to Embodiment 1 described above in that the wireless control device 21 is replaced with a wireless control device 21A. The wireless control device 21A is different from the wireless control device 21 according to Embodiment 1 described above in that the switch 28 is omitted and the wireless control unit 22 is replaced with a wireless control unit 22A (an example of a processing unit).

The wireless control unit 22A determines a posture of the user S, based on the acceleration signal from the acceleration sensor 19 in the same manner as in Embodiment 1 described above. The wireless control unit 22A changes the accuracy of the measurement data of the electrocardiogram sensor 12, based on the posture of the user S. Specifically, the wireless control unit 22A decreases the accuracy of the measurement data of the electrocardiogram sensor 12 by performing interleaving processing for the electrocardiogram signal obtained from the high-performance ADC 16. The interleaving processing may be performed by, for example, at least one of reduction of a resolution of the electrocardiogram signal and resampling of the electrocardiogram signal. By doing so, the wireless control unit 22A transmits measurement data obtained by changing the accuracy in accordance with the posture to the wireless transmission and reception unit 26.

In the same manner as Embodiment 1 described above, the wireless control unit 22A preferably changes the accuracy of the measurement data of the electrocardiogram sensor 12, based on learning information about a posture of the user S in a predetermined time zone in addition to the posture of the user S. Thereby, it is possible to achieve both an increase in opportunity to make a high-accuracy measurement and improvement in efficiency of power consumption, as described above.

In this way, in Embodiment 2, the “high-accuracy measurement” in Embodiment 1 described above is realized as the wireless control unit 22A does not perform the interleaving processing, and the “low-accuracy measurement” in Embodiment 1 is realized as the wireless control unit 22A performs the interleaving processing.

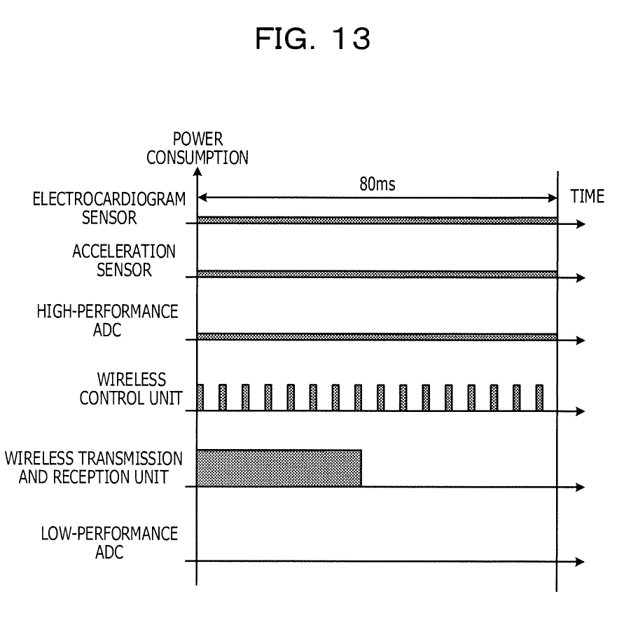

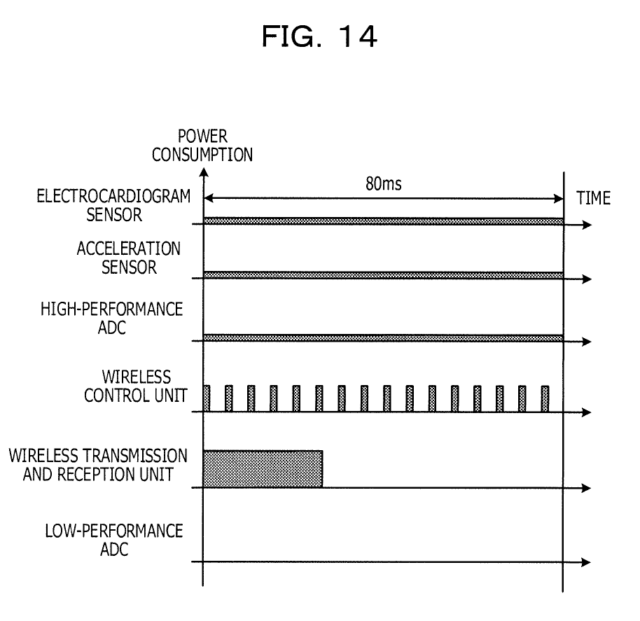

FIG. 13 is a diagram schematically illustrating a time series of power consumption of the electrocardiogram sensor 12 and the like during a high-accuracy measurement, and FIG. 14 is a diagram schematically illustrating the time series of the power consumption of the electrocardiogram sensor 12 and the like during a low-accuracy measurement.

In the examples illustrated in FIGS. 13 and 14, the electrocardiogram signal is converted into a digital signal with 16 samples×24 bits (3 bytes) by the high-performance ADC 16 during a measurement period of 80 ms.

During the high-accuracy measurement (FIG. 13), the electrocardiogram signal from the high-performance ADC 16 is not subjected to interleaving processing in the wireless control unit 22A. Accordingly, measurement data based on the electrocardiogram signal from the high-performance ADC 16 is transmitted as three packets. In contrast to this, during the low-accuracy measurement (FIG. 14), the electrocardiogram signal from the high-performance ADC 16 is subjected to the interleaving processing in the wireless control unit 22A. For example, in a case where the interleaving processing for resampling at 100 Hz is performed, the number of packets is reduced from 3 to 1.5 (≈2). In addition, for example, in a case where the interleaving processing for cutting down to accuracy of 16 bits is performed, the number of packets is reduced from 3 to 2. In this way, in the wireless transmission and reception unit 26, power consumption in the high-accuracy measurement is also increased because the number of packets to be transmitted is larger than in the low-accuracy measurement.

The operation examples (including the third to sixth modification examples described above) described with reference to FIGS. 6 to 10 in Embodiment 1 are also applied to Embodiment 2 in the same manner. However, as described above, in Embodiment 2, the “high-accuracy measurement mode” in Embodiment 1 corresponds to the mode in which the interleaving processing is not performed, and the “low-accuracy measurement mode” in Embodiment 1 corresponds to the mode in which the interleaving processing is performed.

The same effect as in Embodiment 1 described above is also obtained in Embodiment 2. According to Embodiment 2, a hardware configuration can be simplified as compared with Embodiment 1 described above, as can be seen from a comparison between FIG. 2 and FIG. 12. In Embodiment 2, modification examples such as the first modification example and the second modification example in Embodiment 1 described above can also be used. In Embodiment 2, prohibition of measurement according to the first modification example is actually equivalent to a fact that a sampling frequency of a measurement value of the electrocardiogram sensor 12 is lower than a frequency corresponding to one time zone. Alternatively, in Embodiment 2, the prohibition of measurement according to the first modification example is actually equivalent to a fact that a resolution of the measurement value of the electrocardiogram sensor 12 is reduced to zero bit.

### Embodiment 3

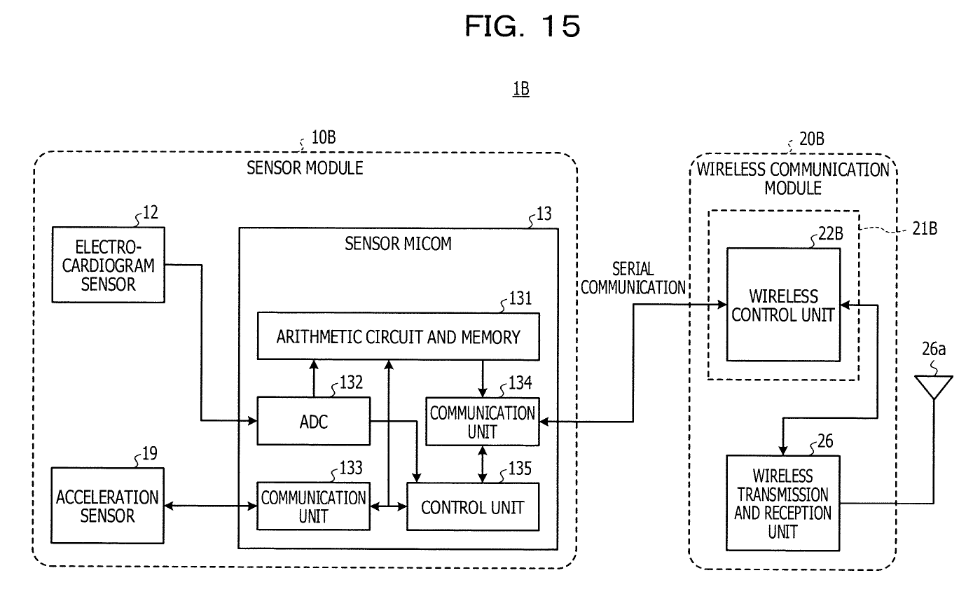

FIG. 15 is a diagram illustrating a configuration example of an electrocardiogram measurement apparatus 1B (an example of a measurement apparatus) according to Embodiment 2. The electrocardiogram measurement apparatus 1B includes a sensor module 10B and a wireless communication module 20B.

The sensor module 10B is different from the sensor module 10 according to Embodiment 1 described above in that the switch 14, the high-performance ADC 16, and the low-performance ADC 18 are replaced with a microcomputer 13. The microcomputer 13 is hereinafter referred to as a “sensor micom 13”. The sensor micom 13 includes an arithmetic circuit and memory 131, an ADC 132 (hereinafter, referred to as an “ADC 132”), communication units 133 and 134, and a control unit 135 (an example of a processing unit).

A storage unit of the arithmetic circuit and memory 131 realizes the learning information storage unit 24 that stores the learning information described above.

The low-performance ADC 132 may have the same configuration as the above-described high-performance ADC 16. The low-performance ADC 132 provides the control unit 135 with an electrocardiogram signal converted into a digital format.

The communication unit 133 acquires an acceleration signal from the acceleration sensor 19 through a serial communication and provides the acquired acceleration signal to the control unit 135.

The communication unit 134 transmits the data obtained from the control unit 135 or the like to the wireless communication module 20B through a serial communication.

The control unit 135 performs the same function as the wireless control unit 22A according to Embodiment 2 described above. That is, the control unit 135 determines a posture of the user S, based on the acceleration signal from the acceleration sensor 19. The control unit 135 changes accuracy of measurement data of the electrocardiogram sensor 12 through the interleaving processing, based on the posture of the user S. In the same manner, the control unit 135 preferably changes the accuracy of the measurement data of the electrocardiogram sensor 12, based on learning information on the posture of the user S in a predetermined time zone (a plurality of times zones can be used), in addition to the posture of the user S.

In this way, in Embodiment 3, the “high-accuracy measurement” in Embodiment 1 is realized as the control unit 135 does not perform the interleaving processing, and the “low-accuracy measurement” is realized as the control unit 135 performs the interleaving processing.

The wireless communication module 20B is different from the wireless communication module 20 according to Embodiment 1 in that the wireless control device 21 is replaced with a wireless control device 21B. The wireless control device 21B is different from the wireless control device 21 according to Embodiment 1 described above in that the switch 28 and the learning information storage unit 24 do not exist and the wireless control unit 22 is replaced with the wireless control unit 22B.

The wireless control unit 22B transmits the measurement data of the electrocardiogram sensor 12 obtained from the sensor module 10B to the wireless transmission and reception unit 26.

The operation examples (including the third to sixth modification examples described above) described with reference to FIGS. 6 to 10 in Embodiment 1 are also applied to Embodiment 3 in the same manner. However, as described above, in Embodiment 3, the “high-accuracy measurement mode” in Embodiment 1 corresponds to the mode in which the interleaving processing is not performed, and the “low-accuracy measurement mode” in Embodiment 1 corresponds to the mode in which the interleaving processing is performed.

The same effect as in Embodiment 1 described above is also obtained in Embodiment 3. According to Embodiment 3, the sensor micom 13 is included, and thereby, a processing load of the wireless control unit 22 can be reduced compared with Embodiment 1 described above. In Embodiment 3, modification examples such as the first modification example and the second modification example in Embodiment 1 described above can also be used. In Embodiment 3, prohibition of measurement according to the first modification example is actually equivalent to a fact that a sampling frequency of a measurement value of the electrocardiogram sensor 12 is lower than a frequency corresponding to one time zone. Alternatively, in Embodiment 3, the prohibition of measurement according to the first modification example is actually equivalent to a fact that a resolution of the measurement value of the electrocardiogram sensor 12 is reduced to zero bit.

As described above, although the respective embodiments are described in detail, the present disclosure is not limited to a specific embodiment, various modifications and changes can be made within a range described in the scope of claims. In addition, it is also possible to combine all or some of the configuration elements of the embodiments described above.

For example, in Embodiment 1 to Embodiment 3 described above, an electrocardiogram is a biological signal to be measured, but other biological signals (for example, an electrograph, an electromyogram, a pulse wave, or the like) may be a person to be measured. In addition, various kinds of biological information (for example, a heartbeat, a pulse, and the like) may be acquired based on the measured biological signal.

In addition, in Embodiment 1 to Embodiment 3 described above, the acceleration sensor 19 is used to detect a posture of the user S, but other sensors (for example, a gyro sensor, an image sensor, and the like) may be used instead of the acceleration sensor 19 or in addition to the acceleration sensor 19. For example, in a case where an image sensor is used, it is possible to determine whether or not the user S is in a stable posture by image recognition, based on an image of a plurality of frames.

In addition, in Embodiment 1 to Embodiment 3 described above, learning information in the learning information storage unit 24 is generated (updated) during an operation, but learning information in the learning information storage unit 24 may be acquired from the outside as previously generated information.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a illustrating of the superiority and inferiority of the invention. Although the embodiments of the present invention have been described in detail, it should be understood that the various changes, substitutions, and alterations could be made hereto without departing from the spirit and scope of the invention.

What is claimed is:

1. A measurement apparatus comprising:
a biological sensor configured to measure a biological signal of a person to be measured; and
a processor configured to:
acquire posture information on a posture of the person to be measured; and
change accuracy of measurement data of the biological sensor, based on the acquired posture information.

2. The measurement apparatus according to claim 1, wherein, in a case where the posture of the person to be measured is a stable posture, the processor increases the accuracy of the measurement data of the biological sensor, compared with a case where the posture of the person to be measured is not the stable posture.

3. The measurement apparatus according to claim 1, wherein the accuracy of the measurement data includes at least one of a resolution of a value that is measured by the biological sensor and a sampling frequency.

4. The measurement apparatus according to claim 3, wherein, in a case where the posture information that is acquired in a first time zone satisfies a criterion, the processor increases at least one of the resolution of the value that is measured by the biological sensor in the first time zone and the sampling frequency, compared with a case where the posture information which is acquired in the first time zone does not satisfy the criterion.

5. The measurement apparatus according to claim 4, wherein, in a case where the posture information that is acquired in the first time zone does not satisfy the criterion, the processor prohibits measurement which is performed by the biological sensor.

6. The measurement apparatus according to claim 1, further comprising:
a learning information storage configured to store learning information on the posture of the person to be measured in a past first time zone,
wherein the processor changes the accuracy of the measurement data in a current first time zone, based on the posture information that is acquired in the current first time zone and the learning information.

7. The measurement apparatus according to claim 6, wherein the learning information includes information indicating whether or not the posture information relating to the past first time zone satisfies a criterion.

8. The measurement apparatus according to claim 6, wherein the learning information indicates a probability that the posture information on the past first time zone satisfies a criterion.

9. The measurement apparatus according to claim 8, wherein, in a case where the probability is greater than or equal to a threshold value, the processor increases at least one of a resolution of a value that is measured by the biological sensor in the current first time zone and a sampling frequency, compared with a case where the probability is less than the threshold value.

10. The measurement apparatus according to claim 9, wherein, in a case where the probability is less than the threshold value, the processor prohibits measurement that is performed by the biological sensor.

11. The measurement apparatus according to claim 8, wherein, in a case where the probability is greater than or equal to a threshold value and the posture information that is acquired in the current first time zone satisfies the criterion, the processor increases at least one of a resolution of a value that is measured by the biological sensor in the current first time zone and a sampling frequency, compared with a case where the probability is not greater than the threshold value and the posture information that is acquired in the current first time zone does not satisfy the criterion.

12. The measurement apparatus according to claim 4, wherein the first time zone is a time zone that is designated by the person to be measured.

13. The measurement apparatus according to claim 7, wherein the processor updates the learning information, based on a result of determination on whether or not the posture information that is acquired in the current first time zone satisfies the criterion.

14. The measurement apparatus according to claim 1, further comprising:
a first analog-digital converter configured to convert an analog signal of the biological sensor into a digital signal; and
a second analog-digital converter configured to convert the analog signal of the biological sensor into the digital signal in a state where at least one of a resolution and a sampling frequency is lower than a resolution and a sampling frequency of the first analog-digital converter,
wherein the processor is coupled to the first analog-digital converter and the second analog-digital converter, and
wherein the accuracy of the measurement data includes a selectively use of the first analog-digital converter or the second analog-digital converter.

15. The measurement apparatus according to claim 1, wherein the biological signal is an electrocardiogram.

16. The measurement apparatus according to claim 1,
wherein the accuracy of the measurement data includes selectively use of a measurement mode of the biological sensor between a first measurement mode in which the biological sensor measures a first biological signal of the person to be measured and a second measurement mode in which the biological sensor measures a second biological signal which has a smaller amplitude than the first biological signal, in addition to the first biological signal.

17. The measurement apparatus according to claim 16, wherein the first biological signal is an electrocardiogram of the person to be measured and the second biological signal is an electrocardiogram of a fetus of the person to be measured.

18. The measurement apparatus according to claim 1, further comprising:
a posture sensor configured to detect the posture of the person to be measured,
wherein the processor acquires the posture information from the posture sensor.

19. The measurement apparatus according to claim 4, wherein the criterion is set based on the posture information when the posture of the person to be measured is a stable posture.

20. A non-transitory computer-readable recording medium recording a measurement program which causes a computer to execute a process, the process comprising:
acquiring posture information on a posture of a person to be measured; and
changing accuracy of measurement data of a biological sensor configured to measure a biological signal of the person to be measured, based on the acquired posture information.
